(19) United States
(12) Patent Application Publication
(10) Pub. No.: US 2026/0345799 A1
(43) Pub. Date: Nov. 5, 2026

(54) SYSTEMS AND METHODS FOR TARGETED DEGRADATION OF INTRACELLULAR PATHOLOGICAL PROTEINS

(71) Applicant: WHITAKER SYNDICATES, LLC, Logan, UT (US)

(72) Inventor: James B. Whitaker, Logan, UT (US)

(73) Assignee: WHITAKER SYNDICATES, LLC, Logan, UT (US)

(21) Appl. No.: 19/665,489

(22) Filed: May 1, 2026

Related U.S. Application Data
(60) Provisional application No. 63/799,371, filed on May 3, 2025.

(51) Int. Cl. A61K 47/62; A61K 47/54

(52) U.S. Cl. CPC A61K 47/62; A61K 47/544

(57) ABSTRACT

According to some implementations of the described systems and methods, one or more therapeutic conjugates are provided. In some implementations, the conjugate includes one or more: peptide backbones; shuttles (e.g., configured to cause the therapeutic conjugate to cross a blood-brain barrier of a patient); tau-targeting ligands (e.g., configured to bind to one or more tau proteins); and tagging domains (e.g., configured to mark the therapeutic conjugate for degradation after the tau-targeting ligand has bound to the tau protein).

### RELATED APPLICATIONS AND PRIORITY CLAIM

This application claims priority to United States Provisional Application No. 63/799,371 entitled “Choline Mimic Peptide Lipid Conjugates for Selective Tau Inhibition and Inducible Lysosomal Clearance Across the Blood Brain Barrier” (Attorney Docket No. 38792.3), filed May 3, 2025, the entire disclosure of which is incorporated herein by reference.

### FIELD

The present disclosure relates to medicine, and more particularly to targeting specific diseases such as tauopathies (e.g., Alzheimer’s disease) with therapeutic conjugates.

### BACKGROUND

Neurodegenerative disorders are a leading cause of morbidity and mortality worldwide and are characterized by progressive loss of neuronal structure and function. Such disorders include, without limitation, Alzheimer’s disease, frontotemporal dementia, chronic traumatic encephalopathy, and other tauopathies, each of which may involve cognitive decline, memory impairment, behavioral changes, and loss of motor function. In many such conditions, disease progression is associated with the accumulation of pathological protein species within the central nervous system.

In addition to tauopathies, a range of related neurological conditions may be associated with similar or overlapping pathological proteins, such as alpha-synuclein in Parkinson’s disease and Lewy Body Dementia (LBD), TDP-43 in Amyotrophic Lateral Sclerosis (ALS) and Frontotemporal Dementia (FTD), and other protein aggregation disorders. These conditions can involve the buildup of misfolded or aggregated proteins, neuroinflammation, synaptic dysfunction, and neuronal degeneration, often resulting in progressive and irreversible neurological impairment.

Neurodegenerative and neurological disorders may also include, in some cases, traumatic brain injury, long-COVID-related neurological sequelae, vascular dementia, and other conditions affecting the central nervous system. Collectively, these disorders represent a significant unmet medical need, as current treatment options are often limited and primarily focused on symptomatic management rather than addressing underlying disease processes.

Thus, while some techniques currently exist that are used to attempt to treat tauopathies and other disorders, challenges still exist, including those listed above. Accordingly, it would be an improvement in the art to augment or even replace current techniques with other techniques.

### SUMMARY

The present disclosure relates generally to systems and methods for treating neurological conditions, including tauopathies, using therapeutic conjugates. In some embodiments, the disclosure provides compositions and methods configured to selectively target pathological protein species (e.g., tau proteins) within the central nervous system and to facilitate degradation or clearance of such species.

According to some embodiments, a therapeutic conjugate is provided. In some embodiments, the conjugate includes one or more components configured to facilitate transport across a biological barrier (e.g., a blood-brain barrier (BBB)), one or more components configured to bind to a target molecule (e.g., a tau protein), and one or more components configured to facilitate degradation or clearance of the target molecule. In some embodiments, the conjugate includes a shuttle configured to facilitate localization of the conjugate at or near a blood–brain barrier and to promote its uptake via endogenous transport or trafficking pathways, a backbone configured to couple one or more components of the conjugate, a tau-targeting ligand configured to bind to tau proteins, and a tagging domain configured to facilitate degradation or clearance of the tau proteins.

In some embodiments, the shuttle includes one or more structures configured to engage one or more transport mechanisms, including carrier-mediated or receptor-mediated transport systems. In some embodiments, the shuttle includes one or more choline-mimicking structures, phosphocholine analogs, glucose-mimicking structures (e.g., headgroups, GLUT1, etc.), peptide-based transport sequences, or other structures configured to facilitate transport of the conjugate across the BBB.

In some embodiments, the tau-targeting ligand includes one or more small-molecule ligands configured to bind to pathological forms of tau, including aggregated, misfolded, or hyperphosphorylated tau. In some embodiments, the tau-targeting ligand exhibits a binding affinity for pathological tau that is greater than a binding affinity for non-pathological tau. In some embodiments, the tau-targeting ligand includes one or more heterocyclic compounds, fused-ring structures, or other structures configured to interact with beta-sheet-rich regions of tau aggregates.

In some embodiments, the tagging domain includes one or more motifs configured to facilitate intracellular routing and degradation of target molecules. In some embodiments, the tagging domain includes one or more lysosomal targeting motifs, including inducible lysosomal targeting motifs (iLTMs), configured to direct a conjugate–target complex to one or more degradation pathways. In some embodiments, the tagging domain is configured to be masked prior to activation and to be activated following one or more triggering events, including binding of the conjugate to a target molecule or exposure to one or more intracellular conditions.

In some embodiments, binding of the tau-targeting ligand to a tau protein facilitates activation of the tagging domain, thereby promoting degradation or clearance of the tau protein. In some embodiments, the tagging domain facilitates lysosomal, endosomal, autophagic, or other intracellular processing of the conjugate–target complex.

According to some embodiments, methods are provided for treating one or more neurological conditions. In some embodiments, the methods include administering a therapeutic conjugate to a subject, transporting the conjugate across the BBB, binding the conjugate to a target molecule (e.g., a pathological tau protein), and facilitating degradation or clearance of the target molecule. In some embodiments, the methods include treating one or more tauopathies, including Alzheimer’s disease, frontotemporal dementia, or other conditions associated with pathological tau.

In some embodiments, the described systems and methods provide advantages over prior approaches by integrating transport, targeting, and degradation functionalities within a single conjugate. In some embodiments, the described systems and methods enable selective targeting and clearance of intracellular aggregates, thereby addressing limitations associated with therapies that do not effectively engage intracellular aggregated protein targets.

### DESCRIPTION OF THE FIGURES

The objects and features of the present disclosure will become more fully apparent from the following description and appended claims, taken in conjunction with the accompanying figures. Understanding that these figures depict only some embodiments of the disclosed systems and methods and are, therefore, not to be considered limiting in scope, the systems and methods will be described and explained with additional specificity and detail through the use of the accompanying figures in which:

FIG. 1 shows a schematic representation of a therapeutic conjugate in accordance with some embodiments of the disclosed systems and methods;

FIG. 2 shows a structural representation of a therapeutic conjugate in accordance with some embodiments; and

FIG. 3 shows a schematic flow diagram illustrating some features of a therapeutic conjugate in accordance with some embodiments.

### DETAILED DESCRIPTION

A description of embodiments will now be given with reference to the Figures. It is expected that the present systems and methods may take many other forms and shapes, hence the following disclosure is intended to be illustrative and not limiting, and the scope of the disclosure should be determined by reference to the appended claims.

The instant systems and methods are directed at various embodiments of therapeutic conjugates (conjugates) 20. Indeed, some embodiments include one or more therapeutic conjugates for selective tau inhibition and inducible lysosomal clearance, along with shuttling capabilities for crossing the blood-brain barrier (BBB). Accordingly, in some embodiments, the conjugate includes one or more: backbones 22 (e.g., peptide backbones); shuttles 24 (e.g., phosphocholine analogs or other transport domains configured to promote interaction with blood–brain barrier (BBB) transport or recognition systems, thereby facilitating delivery across the blood–brain barrier (BBB) via one or more of transporter-mediated shuttling, endothelial uptake, or absorptive translocation.); tau-targeting ligands 26 (e.g., moieties configured to bind pathological proteins such that the pathological proteins affiliate with the conjugate); or tagging domains 28 (e.g., domains configured to selectively facilitate lysosomal degradation or other clearing of the conjugate-tau complex.

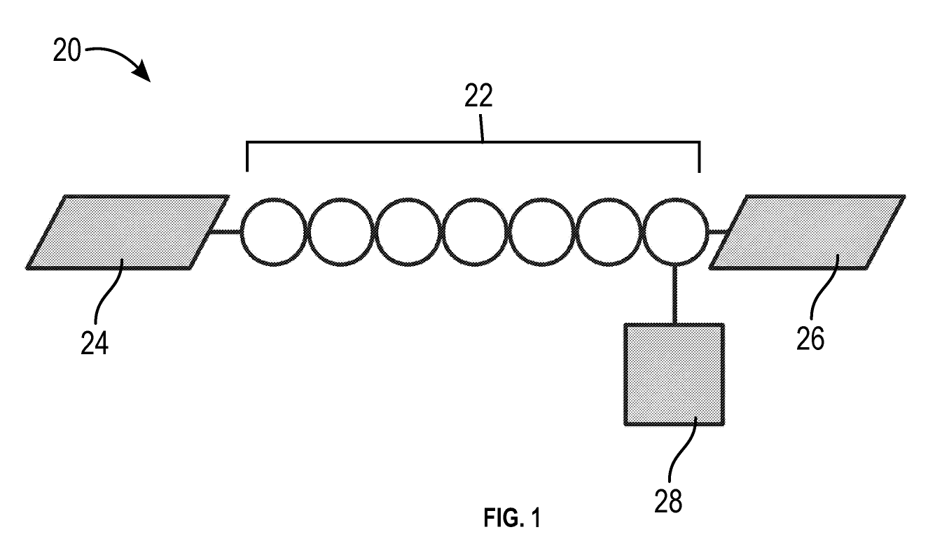

In some embodiments, the conjugate 20 includes one or more backbones 22. The backbone can include any component configured to couple one or more portions of the conjugate together, to provide structural support for the conjugate, to define spatial relationships between one or more domains of the conjugate, or to otherwise facilitate proper positioning and interaction of the various components of the conjugate during use. In some embodiments, the backbone is configured to maintain a separation distance, orientation, or relative flexibility between the shuttle 24, the tau-targeting ligand 26, and the tagging domain 28, such that each component can perform its intended function without substantial steric hindrance or functional interference, and to facilitate coordinated or cooperative function of the shuttle, tau-targeting ligand, and tagging domain. In some embodiments, the backbone further facilitates presentation, exposure, or activation of one or more functional domains in response to target engagement. By way of non-limiting illustration, FIG. 1 shows some embodiments of a conjugate 20 having a backbone 22 extending between a shuttle 24, a tau-targeting ligand 26, and a tagging domain 28.

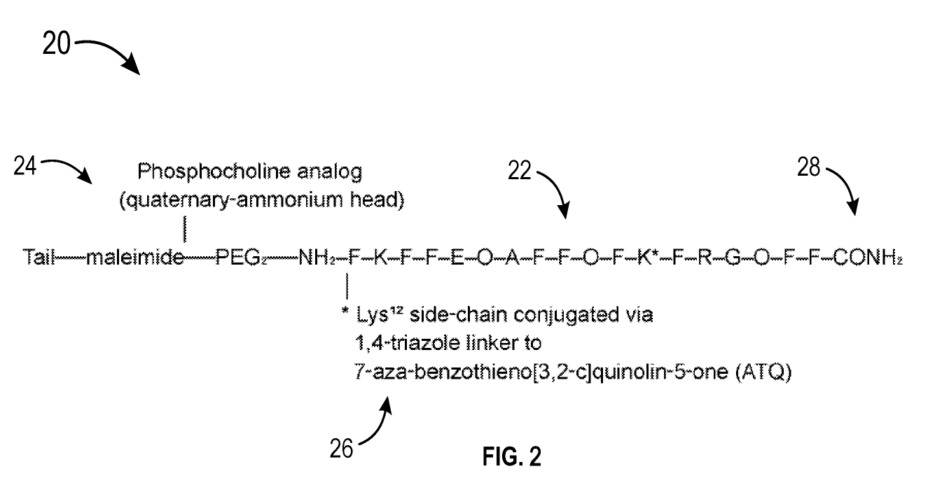

In some embodiments, the backbone 22 includes one or more peptide backbones. The peptide backbone can include any suitable sequence of amino acids configured to provide a desired structural, functional, or biochemical property. For example, in some embodiments, the peptide backbone includes between about 5 and about 100 amino acids, or any subrange therein (e.g., between about 10 and about 50 amino acids, between about 15 and about 30 amino acids, or any other suitable range). As a specific example, some embodiments of the peptide backbone include between 10 and 20 amino acids, the length of the peptide backbone being selected to provide a desired spatial separation and functional interaction between domains. In some embodiments, the peptide backbone includes one or more natural amino acids, non-natural amino acids, modified amino acids, D-amino acids, L-amino acids, or any combination thereof. In some embodiments, the peptide backbone includes one or more amino acids selected to resist proteolytic degradation prior to reaching a target site (e.g., within the central nervous system), to increase stability in physiological environments, or to prolong circulation time of the conjugate within a subject. By way of non-limiting illustration, FIG. 2 shows some embodiments of a conjugate 20 having a peptide backbone 22 configured to couple multiple functional domains of the conjugate.

In some embodiments, the backbone 22 includes one or more stabilizing features configured to enhance resistance to enzymatic degradation. For example, in some embodiments, the backbone includes one or more non-natural amino acids, such as fluorinated amino acids (e.g., 4-fluorophenylalanine), methylated amino acids, cyclized residues, or other modified residues. In some embodiments, the backbone includes a percentage of non-natural amino acids sufficient to reduce susceptibility to proteases, such as at least about 5%, 10%, 15%, 20%, 25%, 30%, or any other suitable percentage of non-natural residues. In some embodiments, the backbone includes structural features such as cyclization, stapling, crosslinking, or other conformational constraints configured to stabilize the backbone. By way of non-limiting illustration, FIG. 2 shows some embodiments of a backbone 22 including one or more modified amino acid residues configured to enhance stability. In this regard, FIG. 2, which provides a non-limiting example of a conjugate in accordance with this disclosure, is intended to be interpreted as follows: F = L-Phenylalanine; O = 4-Fluoro-L-Phenylalanine (a non-naturally-occurring residue); the N-terminus is capped with a maleimide-PEG2 linker bound to a phosphocholine-analog lipid tail for CTL1/CTL2 transport; the C-terminus is amidated and extended by Val-Gly-Leu-Gly-Asp (iLTM) masked by a Val-Gly cathepsin-labile spacer; single-letter amino-acid codes are used for natural residues (the unnatural residue 4-F-Phe is designated as “O” per INSDC guidelines).

In some embodiments, the backbone 22 is configured to include one or more coupling sites for attachment of other components of the conjugate 20. The coupling sites can include any suitable functional groups or reactive sites, such as amines, carboxyl groups, thiols, azides, alkynes, maleimides, or any other suitable chemical functionalities. In some embodiments, the backbone is configured to couple to one or more components via covalent bonds, including amide bonds, ester bonds, thioether bonds, triazole linkages, click chemistry linkages, or any other suitable coupling mechanisms. In some embodiments, the backbone is configured to permit selective coupling at predetermined positions along the backbone, including positioning of one or more domains at an N-terminus, C-terminus, or intermediate location along the backbone, such that the relative placement of the shuttle 24, the tau-targeting ligand 26, and the tagging domain 28 can be controlled. By way of non-limiting illustration, FIG. 2 shows some embodiments of a backbone 22 having one or more coupling sites configured to attach to other components of the conjugate 20.

In some embodiments, the backbone 22 can have any suitable geometry, configuration, or construction. For example, in some embodiments, the backbone is substantially linear, branched, cyclic, helical, or otherwise configured. In some embodiments, the backbone is flexible, semi-rigid, or rigid, depending on the desired spacing and interaction between components of the conjugate. In some embodiments, the backbone includes one or more spacer regions, linker segments, or flexible domains configured to provide mobility between components, or one or more rigid segments configured to maintain defined spatial relationships, and can further include one or more non-peptidic linker segments (e.g., polyethylene glycol (PEG) linkers or other spacer chemistries). By way of non-limiting illustration, FIG. 1 shows some embodiments of a backbone 22 configured to extend between multiple domains of the conjugate 20 in a generally linear configuration.

In accordance with some embodiments, the conjugate 20 includes one or more shuttles 24. The shuttle can include any component configured to facilitate transport of the conjugate across a biological barrier, such as the blood-brain barrier (BBB), to enable delivery of the conjugate to a target site within the central nervous system (or elsewhere). In some embodiments, the shuttle is configured to promote uptake of the conjugate into brain tissue by engaging one or more endogenous transport mechanisms, thereby increasing the concentration of the conjugate at or near neuronal cells relative to systemic circulation. By way of non-limiting illustration, FIG. 1 shows some embodiments of a conjugate 20 having a shuttle 24 coupled to a backbone 22 and configured to facilitate transport of the conjugate across the BBB.

In some embodiments, the shuttle 24 includes one or more structures configured to mimic or otherwise interact with naturally transported substrates of the BBB. Indeed, in some embodiments, the shuttle is configured to structurally and/or functionally resemble a naturally occurring nutrient, metabolite, or signaling molecule, such that the shuttle engages a corresponding transporter and is transported across the BBB in a manner analogous to the endogenous substrate. In some embodiments, the shuttle is configured to exploit nutrient transport pathways, including those associated with choline transport, glucose transport, amino acid transport, or other carrier-mediated systems, thereby facilitating uptake of the conjugate into the central nervous system via active transport mechanisms rather than passive diffusion. For example, some embodiments of the shuttle include one or more choline-mimicking structures configured to engage one or more choline transporters (e.g., CTL1, CTL2, or other transporters) expressed at the BBB. In some embodiments, the shuttle includes a phosphocholine analog or other quaternary ammonium-containing structure configured to resemble endogenous choline or choline-containing compounds, thereby facilitating active transport across the BBB. In some embodiments, the shuttle includes one or more lipid-like moieties (e.g., C₁₂-C₂₄ alkyl chains, C₁₄-C₂₀ alkyl chains, or any other suitable lipid-like moieties) configured to enhance interaction with cellular membranes or transporter systems. By way of non-limiting illustration, FIG. 2 shows some embodiments of a shuttle 24 comprising a phosphocholine analog and a tail (e.g., a lipid-like moiety) coupled to a backbone 22 via a linker.

In some embodiments, the shuttle 24 is configured to engage one or more alternative transport pathways. For example, in some embodiments, the shuttle includes one or more glucose-mimicking structures configured to engage glucose transporters (e.g., GLUT1 or other transporters) at the BBB. In some embodiments, the shuttle includes one or more amino acid analogs, peptide-based transport sequences, receptor-binding ligands, or other structures configured to engage receptor-mediated or carrier-mediated transport systems. In some embodiments, the shuttle includes one or more peptide sequences configured to bind to receptors such as low-density lipoprotein receptor-related protein (LRP1), transferrin receptor (TfR), insulin receptor, or other receptors involved in transcytosis across the BBB. By way of non-limiting illustration, FIG. 1 shows some embodiments of a conjugate 20 in which a shuttle 24 is configured to engage a transport mechanism to facilitate movement of the conjugate into brain tissue.

In some embodiments, the shuttle 24 is configured to function in combination with other properties of the conjugate to enhance delivery across the BBB. For example, in some embodiments, the shuttle is configured to cooperate with physicochemical properties of the backbone 22 (e.g., hydrophobicity, charge distribution, or molecular size) to facilitate uptake and retention within brain tissue. In some embodiments, the shuttle is configured to provide sufficient affinity for a transport mechanism to enable uptake, while allowing release of the conjugate once across the BBB. In some embodiments, the shuttle is configured to balance transport efficiency with systemic stability, thereby avoiding premature clearance or degradation prior to reaching the target site. By way of non-limiting illustration, FIG. 2 shows some embodiments of a conjugate 20 in which a shuttle 24 is coupled to a backbone 22 and positioned to facilitate interaction with a transport system while maintaining overall structural integrity of the conjugate.

Where the shuttle 24 includes one or more phosphocholine analogs, the phosphocholine analog can include any component configured to engage a choline transporter (e.g., CTL1, CTL2, or another choline transporter) expressed at the BBB. For example, the phosphocholine analog can include one or more: phosphorylcholine headgroups; phosphatidylcholine derivatives; lysophosphatidylcholine (LPC) analogs; glycerophosphorylcholine (GPC) analogs; alkylphosphocholine compounds (e.g., erucylphosphocholine, miltefosine, or other C₁₂-C₂₄ alkylphosphocholine derivatives); platelet-activating factor (PAF) analogs; synthetic phosphocholine-containing lipids; phosphocholine-functionalized polymers; phosphocholine-modified small molecules; quaternary ammonium-containing choline mimetics (e.g., trimethylammonium-containing compounds structurally analogous to choline); fluorinated choline analogs (e.g., fluoroalkyl choline derivatives); deuterated choline analogs; choline kinase substrates or analogs thereof; bioorthogonal phosphocholine analogs configured for membrane incorporation; phosphocholine-containing prodrugs; or any other compound comprising a choline-mimetic headgroup configured to engage choline transport systems. In some embodiments, the phosphocholine analog includes a hydrophobic tail portion (e.g., one or more alkyl chains, lipid chains, or amphiphilic moieties) coupled to a polar phosphocholine headgroup, such that the analog mimics endogenous phospholipids and facilitates transporter-mediated uptake and/or membrane interaction.

In some embodiments, the shuttle 24 is configured to provide a balance between affinity for a transport mechanism and release of the conjugate following transport across the BBB. For example, in some embodiments, the shuttle is configured to bind to a transporter with sufficient affinity to enable uptake into or across BBB-associated cells, while permitting dissociation of the conjugate from the transporter following translocation, such that the conjugate can distribute within brain tissue and interact with one or more target molecules. In some embodiments, the shuttle is configured to avoid irreversible binding to a transporter or sequestration within endothelial cells, thereby promoting effective delivery of the conjugate to neuronal or other target cells.

In some embodiments, the shuttle 24 is configured to facilitate transport of a conjugate having a molecular size greater than that of typical endogenous transporter substrates. In some embodiments, the shuttle is configured to engage a transport mechanism in a manner that accommodates or tolerates attachment of the conjugate to the shuttle, such that the conjugate can be transported across the BBB despite having a molecular weight or structural complexity greater than that of the corresponding endogenous substrate. In some embodiments, the shuttle is configured such that one or more portions of the conjugate (e.g., the backbone 22, tau-targeting ligand 26, or tagging domain 28) do not substantially interfere with transporter engagement or translocation. By way of non-limiting illustration, FIG. 2 shows some embodiments in which the shuttle 24 is coupled to a multi-domain conjugate 20 while maintaining functionality of the shuttle.

In some embodiments, the shuttle 24 is configured to facilitate delivery of the conjugate following one or more routes of administration, including intranasal administration, sublingual administration, oral administration, intravenous administration, or other suitable routes. In some embodiments, the shuttle is configured to enhance uptake of the conjugate into brain tissue following intranasal delivery, including by facilitating transport across nasal epithelium and/or subsequent transport across the BBB. In some embodiments, the shuttle is configured to maintain functionality following formulation in one or more pharmaceutical compositions.

In some embodiments, the conjugate 20 includes one or more tau-targeting ligands 26. The tau-targeting ligand can include any component configured to bind to tau proteins, including hyperphosphorylated tau, aggregated tau, misfolded tau, oligomeric tau, fibrillar tau, or any other pathological form of tau, such that the pathological tau proteins associate with the conjugate. In some embodiments, the tau-targeting ligand is configured to selectively bind pathological tau relative to non-pathological tau, thereby facilitating targeted interaction with disease-associated tau species. Indeed, in some cases, the binding affinity of the conjugate to hyperphosphorylated tau (or other pathological tau) is at least 25% (or more) greater than the binding affinity for non-pathological tau. For example, in some cases the binding affinity is 50%, 75%, 100%, 150%, 200%, 250%, or any other suitable amount greater for pathological tau than non-pathological tau. By way of non-limiting illustration, FIG. 1 shows some embodiments of a conjugate 20 having a tau-targeting ligand 26 coupled to a backbone 22.

Relatedly, some embodiments of the tau-targeting ligand 26 are specifically configured to target the hyperphosphorylation or to bind via interaction with one or more structural features associated with pathological tau. For example, in some embodiments, the tau-targeting ligand is configured to interact with beta-sheet structures, hydrophobic grooves, stacking interfaces, or other structural motifs present in aggregated or fibrillar tau. In some embodiments, the tau-targeting ligand is configured to preferentially bind tau conformations associated with disease states, including conformations characterized by altered folding, aggregation, or phosphorylation patterns. By way of non-limiting illustration, FIG. 2 shows some embodiments in which the tau-targeting ligand 26 is positioned to interact with structural features of tau proteins.

In some embodiments, the tau-targeting ligand 26 includes one or more small-molecule ligands. The small-molecule ligand can include any suitable compound configured to bind tau proteins with a desired affinity, specificity, or selectivity. In some embodiments, the tau-targeting ligand includes one or more heterocyclic compounds, polycyclic aromatic compounds, fused-ring systems, or other structures configured to interact with beta-sheet-rich regions of tau aggregates. In some embodiments, the tau-targeting ligand includes one or more compounds selected to bind to tau fibrils, paired helical filaments, or other aggregated tau structures. In some embodiments, the tau-targeting ligand exhibits a dissociation constant (KD) of less than about 1 μM, less than about 500 nM, less than about 100 nM, less than about 10 nM, less than about 1 nM, or any other suitable affinity. In some cases, the conjugate may be particularly effective if KD is less than about 100 nM (± 25 nM).

In some embodiments, the tau-targeting ligand 26 includes one or more hetero-tricyclic ligands. For example, in some embodiments, the tau-targeting ligand includes a quinoline-based compound, benzothiazole-based compound, benzothiophene-based compound, or other fused heterocyclic compound. In some embodiments, the tau-targeting ligand includes a 7-aza-benzothieno[3,2-c]quinolin-5-one (ATQ) compound or a derivative thereof. In some embodiments, the tau-targeting ligand includes one or more analogs, derivatives, or structurally related compounds configured to bind tau proteins. In some embodiments, the tau-targeting ligand includes one or more fluorescent ligands, imaging agents, or diagnostic probes adapted for therapeutic use. By way of non-limiting illustration, FIG. 2 shows some embodiments of a conjugate 20 including a hetero-tricyclic tau-targeting ligand 26.

In some embodiments, the tau-targeting ligand 26 is configured to be coupled to the backbone 22 at one or more positions. In some embodiments, the tau-targeting ligand is coupled to a side chain of an amino acid residue within the backbone (e.g., a lysine residue), such that the ligand extends from the backbone as a branched structure. In some embodiments, the tau-targeting ligand is coupled via one or more linkers, including triazole linkages, click chemistry linkages, amide linkages, thioether linkages, or any other suitable coupling mechanisms. In some embodiments, the tau-targeting ligand is positioned along the backbone to facilitate interaction with tau proteins while minimizing interference with the shuttle 24 and tagging domain 28. By way of non-limiting illustration, FIG. 2 shows some embodiments of a tau-targeting ligand 26 coupled to a lysine side chain of the backbone 22 via a 1,4-triazole linker.

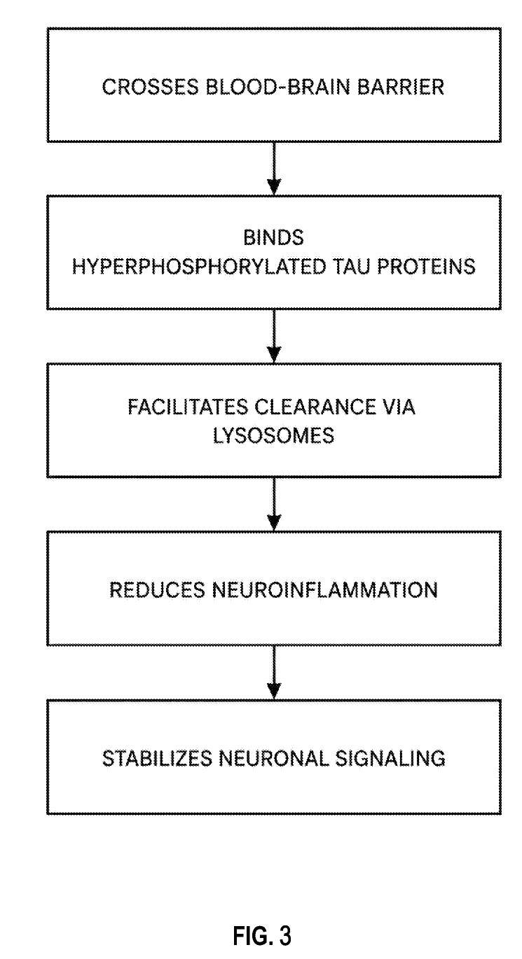

In some embodiments, the tau-targeting ligand 26 is configured to cooperate with other components of the conjugate 20 to facilitate one or more downstream processes. For example, in some embodiments, binding of the tau-targeting ligand to tau proteins positions the tagging domain 28 in proximity to the tau proteins, thereby facilitating activation or exposure of the tagging domain and subsequent targeting of the tau proteins for degradation. In some embodiments, the tau-targeting ligand is configured to bind tau proteins in a manner that promotes internalization, processing, or routing of the conjugate-tau complex within a cell. By way of non-limiting illustration, FIG. 3 shows how in some embodiments, the tau-targeting ligand facilitates association of the conjugate with tau proteins (e.g., hyperphosphorylated tau proteins) prior to downstream processing.

In some embodiments, the tau-targeting ligand 26 is interchangeable with one or more alternative ligands configured to bind different target proteins or different forms of tau. For example, in some embodiments, the tau-targeting ligand is replaced with a ligand configured to bind alpha-synuclein, TDP-43, or other aggregation-prone proteins. In some embodiments, the tau-targeting ligand includes one or more alternative compounds selected to target different tau isoforms, aggregation states, or disease-associated conformations. In some embodiments, the tau-targeting ligand includes one or more ligands selected based on desired affinity, selectivity, or pharmacokinetic properties. By way of non-limiting illustration, FIG. 1 shows some embodiments in which the tau-targeting ligand 26 is configured as part of a modular system permitting substitution with alternative ligands.

In some embodiments, binding of the tau-targeting ligand 26 to tau proteins functions as a triggering event for one or more downstream processes. For example, in some embodiments, binding of the tau-targeting ligand induces a conformational change, spatial repositioning, or exposure of one or more portions of the conjugate, including one or more portions of the tagging domain 28. In some embodiments, binding of the tau-targeting ligand facilitates access of one or more proteases to a cleavable region of the conjugate, thereby enabling activation of the tagging domain. In some embodiments, the tau-targeting ligand is configured such that binding to tau proteins initiates a sequence of events leading to degradation or clearance of the tau proteins. For instance, as discussed in more detail below, in some embodiments, binding of the tau-targeting ligand to tau proteins facilitates subsequent interaction of the tagging domain 28 with the tau proteins. In some cases, this leads to inducible lysosomal degradation or other clearing. By way of non-limiting illustration, FIG. 3 shows some embodiments in which binding of the tau-targeting ligand 26 precedes activation of downstream processes.

According to some embodiments, the conjugate 20 includes one or more tagging domains 28. The tagging domain can include any component configured to facilitate degradation, clearance, sequestration, or processing of one or more target molecules (e.g., tau proteins) associated with the conjugate (either alone or together with the conjugate). In some embodiments, the tagging domain is configured to promote lysosomal degradation of the target molecules, including by directing the conjugate-target complex to one or more intracellular degradation pathways. In some embodiments, the tagging domain is configured to remain inactive or masked until one or more triggering events occur, such as binding of the tau-targeting ligand 26 to tau proteins. By way of non-limiting illustration, FIG. 1 shows some embodiments of a conjugate 20 having a tagging domain 28 coupled to a backbone 22.

In some embodiments, the tagging domain 28 includes one or more lysosomal targeting motifs (e.g., inducible lysosomal targeting motifs, or iLTMs). The lysosomal targeting motif can include any sequence or structure configured to direct a molecule to a lysosome or lysosome-associated pathway. For example, in some embodiments, the tagging domain includes one or more peptide motifs comprising di-leucine sequences, dileucine-based sorting signals, tyrosine-based motifs, KFERQ-like motifs, or other sequences known to facilitate lysosomal targeting or autophagic processing. In some embodiments, the tagging domain includes a di-leucine-containing motif, such as Leu-Leu-Gly-Asp, Val-Gly-Leu-Gly-Asp, or a derivative thereof, configured to facilitate lysosomal routing. By way of non-limiting illustration, FIG. 2 shows some embodiments of a conjugate 20 including a tagging domain 28 comprising a lysosomal targeting motif.

In some embodiments, the tagging domain 28 is masked or otherwise rendered inactive prior to activation. For example, in some embodiments, the tagging domain includes a masking region, blocking group, or steric element configured to prevent interaction of the tagging domain with cellular machinery until activation occurs. In some embodiments, the tagging domain is masked by a cleavable spacer, linker, or sequence configured to be removed under one or more conditions. In some embodiments, the masking region prevents premature degradation or clearance of the conjugate prior to a triggering event (as an example, to prevent degradation of the conjugate 20 until it is bound to tau). By way of non-limiting illustration, FIG. 2 shows some embodiments in which the tagging domain 28 includes a masked lysosomal targeting motif.

In some embodiments, the tagging domain 28 includes one or more cleavable regions configured to be processed by one or more enzymes. For example, in some embodiments, the tagging domain includes one or more protease-cleavable linkers configured to be cleaved by lysosomal or endosomal proteases (e.g., cathepsins). In some embodiments, the tagging domain includes a spacer comprising one or more amino acid sequences such as Val-Gly, Gly-Phe, Gly-Phe-Leu-Gly, or other protease-sensitive sequences. In some embodiments, cleavage of the linker exposes the lysosomal targeting motif, thereby activating the tagging domain. By way of non-limiting illustration, FIG. 2 shows some embodiments in which a cleavable spacer is positioned upstream of a lysosomal targeting motif within the tagging domain 28.

In some embodiments, the tagging domain 28 is configured to cooperate with other components of the conjugate 20 to facilitate targeted degradation. For example, in some embodiments, binding of the tau-targeting ligand 26 to tau proteins positions the tagging domain in proximity to the tau proteins (in some cases, allowing for or recruiting protease to cleave the linker), thereby enabling activation of the tagging domain and subsequent routing of the conjugate-tau complex to lysosomes. In some embodiments, the tagging domain is configured to recruit one or more adaptor proteins, receptors, or cellular components involved in trafficking to lysosomes or autophagosomes. In some embodiments, the tagging domain promotes autophagic uptake, endosomal sorting, or other intracellular processes leading to degradation of the associated target molecules. By way of non-limiting illustration, FIG. 3 shows some embodiments in which the tagging domain facilitates lysosomal clearance following binding of the conjugate to tau proteins.

In some embodiments, the tagging domain 28 is configured to operate in an inducible manner. For example, in some embodiments, activation of the tagging domain (e.g., through cleavage of the linker) occurs only after the conjugate binds to a target molecule, such that degradation is selectively directed to target-associated species. In some embodiments, the tagging domain is configured such that activation depends on one or more conditions present within a cell (e.g., pH, enzymatic activity, intracellular localization) or local to the conjugate 20 (e.g., when the conjugate has bound to a specific target molecule, such as pathological tau—thus, in some cases, if the conjugate binds to a non-target (such as non-pathological tau, in some cases), the tagging domain is not activated). In some embodiments, the tagging domain is configured to minimize off-target degradation by remaining inactive until the conjugate is bound to a target and exposed to an activating environment. By way of non-limiting illustration, FIG. 3 shows some embodiments in which the tagging domain is activated following engagement of the conjugate with tau proteins and subsequent intracellular processing.

In some embodiments, the tagging domain 28 functions as a signaling element configured to direct intracellular trafficking of the conjugate-target complex. For example, in some embodiments, the tagging domain includes one or more motifs recognized by adaptor proteins, sorting machinery, or trafficking complexes within a cell. In some embodiments, the tagging domain is configured to interact with one or more cellular components involved in endosomal sorting, lysosomal targeting, or autophagic processing. In some embodiments, exposure of the tagging domain results in recognition of the conjugate-target complex as cargo for degradation pathways. In some embodiments, the tagging domain 28 is configured to facilitate degradation of target molecules via lysosomal or autophagy-associated pathways independent of ubiquitin-mediated proteasomal degradation. For example, in some embodiments, the tagging domain is configured to direct the conjugate-target complex to lysosomes without requiring ubiquitination of the target molecule or recruitment of an E3 ligase. In some embodiments, this provides advantages over proteasome-targeting systems by enabling degradation of aggregation-prone or structurally complex proteins that may not be efficiently processed by the proteasome. By way of non-limiting illustration, FIG. 3 shows some embodiments in which the tagging domain facilitates lysosomal routing of the conjugate-target complex.

In some embodiments, the tagging domain 28 is configured to facilitate routing and degradation of a conjugate-target complex as a unit. For example, in some embodiments, the tagging domain promotes uptake, trafficking, or sequestration of the conjugate together with one or more associated target molecules, such that the complex is directed to intracellular degradation pathways. In some embodiments, this enables removal of aggregated or otherwise difficult-to-process target species by coupling the target to a degradation-directing structure.

In some embodiments, activation of the tagging domain 28 can occur via one or more mechanisms in addition to, or instead of, protease-mediated cleavage. For example, in some embodiments, activation of the tagging domain is influenced by pH conditions (e.g., acidic environments associated with endosomes or lysosomes), intracellular localization, conformational changes of the conjugate, or exposure of one or more regions of the conjugate following binding to a target molecule. In some embodiments, the tagging domain is configured to respond to one or more environmental or contextual triggers within a cell, thereby enabling selective activation under desired conditions.

In some embodiments, the tagging domain 28 includes one or more non-peptide or hybrid structures configured to facilitate intracellular routing or degradation. For example, in some embodiments, the tagging domain includes one or more small-molecule motifs, polymeric structures, or multifunctional constructs configured to interact with intracellular trafficking machinery. In some embodiments, the tagging domain includes one or more elements configured to recruit adaptor proteins, receptors, or other cellular components associated with degradation pathways. In some embodiments, the tagging domain is configured as part of a modular system in which different tagging strategies can be employed depending on a desired degradation pathway or target molecule.

In some embodiments, one or more methods are provided for treating one or more neurological conditions, including tauopathies, using embodiments of the conjugate 20 described herein. In some embodiments, the method includes administering a therapeutically effective amount of the conjugate to a subject to facilitate transporting the conjugate across a biological barrier (e.g., the blood-brain barrier (BBB)) via the shuttle 24, binding the conjugate to one or more target molecules (e.g., pathological tau proteins) via the tau-targeting ligand 26, and facilitating degradation, clearance, or processing of the target molecules via the tagging domain 28. In some embodiments, the method includes selectively targeting pathological tau species within the central nervous system while minimizing interaction with non-pathological tau.

In some embodiments, the method includes transporting the conjugate 20 across the BBB via one or more carrier-mediated or receptor-mediated transport mechanisms. For example, in some embodiments, the shuttle 24 engages one or more endogenous transport systems (e.g., choline transporters, glucose transporters, amino acid transporters, or receptor-mediated transcytosis pathways) to facilitate uptake of the conjugate into brain tissue. In some embodiments, the method includes delivering the conjugate to neuronal cells or other cells within the central nervous system following transport across the BBB.

In some embodiments, the method includes binding the conjugate 20 to one or more pathological tau proteins via the tau-targeting ligand 26. In some embodiments, the tau-targeting ligand selectively binds hyperphosphorylated, aggregated, or otherwise pathological tau, thereby forming a conjugate–target complex. In some embodiments, the method includes positioning the tagging domain 28 in proximity to the tau proteins as a result of binding of the tau-targeting ligand.

In some embodiments, the method includes activating the tagging domain 28 following binding of the conjugate 20 to a target molecule. For example, in some embodiments, the method includes cleaving a masking region of the tagging domain via one or more enzymes (e.g., cathepsins), thereby exposing a lysosomal targeting motif. In some embodiments, the method includes activating the tagging domain in response to one or more intracellular conditions, including enzymatic activity, pH, or intracellular localization.

In some embodiments, the method includes facilitating intracellular routing and degradation of the conjugate–target complex. For example, in some embodiments, the method includes directing the conjugate–target complex to one or more lysosomal, endosomal, or autophagic pathways, thereby promoting degradation or clearance of the target molecule. In some embodiments, the method includes degrading aggregated or misfolded tau proteins within a cell.

In some embodiments, the method includes administering the conjugate 20 via one or more routes of administration, including intranasal, sublingual, oral, intravenous, or other suitable routes. In some embodiments, the method includes delivering the conjugate in one or more pharmaceutical compositions, formulations, or dosage forms. In some embodiments, the method includes administering the conjugate at a frequency, dosage, or duration sufficient to reduce the presence of pathological tau or to improve one or more symptoms associated with a neurological condition.

In some embodiments, the method includes treating one or more diseases or disorders, including Alzheimer’s disease, frontotemporal dementia, chronic traumatic encephalopathy, or other tauopathies. In some embodiments, the method includes treating one or more additional conditions associated with protein aggregation, including Parkinson’s disease, Lewy body dementia, Huntington’s disease, amyotrophic lateral sclerosis (ALS), traumatic brain injury, or other neurological conditions.

In some embodiments, the method includes modifying one or more components of the conjugate 20 (e.g., the shuttle 24, backbone 22, tau-targeting ligand 26, or tagging domain 28) to target different molecules, pathways, or disease states. In some embodiments, the method includes selecting one or more components of the conjugate based on desired properties, including transport efficiency, binding affinity, selectivity, or degradation pathway. In some embodiments, the method includes using the conjugate as part of a modular platform for targeting and degrading one or more intracellular proteins.

According to some embodiments, one or more methods of administration of the conjugate 20 are provided. In some embodiments, the method includes administering the conjugate to a subject in need thereof, including a subject suffering from one or more neurological conditions, such as a tauopathy or another disorder associated with pathological protein aggregation. In some embodiments, the conjugate is administered in an amount effective to facilitate transport of the conjugate across the blood-brain barrier, binding of the conjugate to one or more target molecules (e.g., pathological tau), and degradation, clearance, or other processing of the target molecules. In some embodiments, the conjugate is administered in one or more pharmaceutical formulations configured to facilitate delivery to the central nervous system. By way of non-limiting illustration, FIG. 3 shows some embodiments in which the conjugate 20 is administered and subsequently transported, bound to target molecules, and processed.

In some embodiments, the conjugate 20 is administered intranasally. For example, in some embodiments, the conjugate is provided as an intranasal spray formulation. In some embodiments, the intranasal spray includes the conjugate at a concentration of about 10 mg mL-1 in an isotonic citrate buffer. In some embodiments, the citrate buffer has a pH of about 5.8. In some embodiments, the intranasal spray formulation is configured to facilitate uptake of the conjugate across nasal epithelium and/or subsequent transport of the conjugate across the blood-brain barrier. In some embodiments, the intranasal spray is configured to provide localized delivery to nasal tissues together with systemic and/or central nervous system exposure. By way of non-limiting illustration, some embodiments include Formulation A, comprising about 10 mg mL-1 of the conjugate in isotonic citrate buffer at about pH 5.8 for intranasal administration.

In some embodiments, the conjugate 20 is administered sublingually. For example, in some embodiments, the conjugate is provided in a sublingual microtablet. In some embodiments, the microtablet includes about 2 mg of the conjugate. In some embodiments, the microtablet further includes a carrier, such as beta-cyclodextrin or another cyclodextrin-based carrier, configured to facilitate stability, handling, dissolution, or absorption of the conjugate. In some embodiments, the sublingual microtablet is configured to dissolve or otherwise release the conjugate within the oral cavity for absorption through sublingual tissues. By way of non-limiting illustration, some embodiments include Formulation B, comprising a sublingual microtablet containing about 2 mg of the conjugate together with a beta-cyclodextrin carrier.

In some embodiments, the conjugate 20 is administered according to one or more dosing regimens. For example, in some embodiments, the conjugate is administered once daily, multiple times per day, once every several days, weekly, or according to any other suitable schedule. In some embodiments, the route of administration, concentration, dosage, formulation, and frequency of administration are selected based on one or more factors, such as the condition being treated, the severity of the condition, the properties of the formulation, or patient-specific characteristics. In some embodiments, administration of the conjugate results in delivery of the conjugate to the central nervous system, selective binding of the conjugate to pathological tau, and inducible degradation or clearance of target molecules.

According to some embodiments, one or more methods for synthesizing the conjugate 20 are provided. In some embodiments, the conjugate 20 is synthesized using one or more peptide synthesis and conjugation techniques. For example, in some embodiments, the backbone 22 is synthesized using solid-phase peptide synthesis (SPPS), including fluorenylmethyloxycarbonyl (Fmoc)-based chemistry or any other suitable peptide synthesis methodology. In some embodiments, amino acid residues (including natural and non-natural amino acids) are sequentially coupled to a growing peptide chain on a solid support, followed by deprotection and cleavage from the support to yield a peptide backbone. In some embodiments, one or more modified residues (e.g., fluorinated amino acids, such as 4-fluorophenylalanine) are incorporated during synthesis to provide one or more desired properties (e.g., protease resistance or structural stability). By way of non-limiting illustration, FIG. 2 shows some embodiments of a conjugate 20 including a peptide backbone 22 formed via solid-phase peptide synthesis.

In some embodiments, the tau-targeting ligand 26 is coupled to the backbone 22 via one or more bioorthogonal conjugation reactions. For example, in some embodiments, the backbone includes one or more functionalized amino acid residues (e.g., ε-alkyne-Lys or other alkyne- or azide-functionalized residues) configured to serve as attachment points. In some embodiments, the tau-targeting ligand is coupled to the backbone via a copper-catalyzed azide–alkyne cycloaddition (CuAAC) reaction or other click chemistry reaction, thereby forming a triazole linkage between the ligand and the backbone. In some embodiments, such coupling occurs at a side chain of a residue within the backbone to provide a branched conjugate structure. By way of non-limiting illustration, FIG. 2 shows some embodiments in which the tau-targeting ligand 26 is coupled to the backbone 22 via a triazole linkage.

In some embodiments, the shuttle 24 is coupled to the backbone 22 via one or more chemical coupling reactions. For example, in some embodiments, the shuttle includes a phosphocholine analog or other BBB shuttle moiety coupled to a linker (e.g., a polyethylene glycol (PEG) linker), which is in turn coupled to the backbone. In some embodiments, the shuttle is coupled to the backbone via a maleimide-based coupling reaction, such as reaction of a maleimide-functionalized shuttle with a thiol or other nucleophilic group associated with the backbone (e.g., at an N-terminus or other functionalized site). In some embodiments, the shuttle is coupled to the backbone at a terminal position, such as an N-terminus, to facilitate transport functionality. By way of non-limiting illustration, FIG. 2 shows some embodiments in which the shuttle 24 is coupled to the backbone 22 via a maleimide-PEG linker.

In some embodiments, the tagging domain 28 is formed as part of the backbone 22 during peptide synthesis and/or through subsequent modification steps. For example, in some embodiments, one or more lysosomal targeting motifs (e.g., di-leucine-containing sequences) and one or more cleavable spacer sequences (e.g., protease-sensitive linkers such as Val-Gly) are incorporated into the peptide backbone during solid-phase synthesis. In some embodiments, one or more masking regions are included to provide an inducible tagging domain, as described above.

In some embodiments, following synthesis and conjugation, the conjugate 20 is purified using one or more purification techniques. For example, in some embodiments, the conjugate is purified using reverse-phase high-performance liquid chromatography (RP-HPLC) or other chromatographic techniques. In some embodiments, purification yields a conjugate having a purity of at least about 70%, 80%, 85%, 90%, 95%, 98%, 99%, or any other suitable purity level. In some embodiments, the conjugate is purified to a purity of at least about 95%. In some embodiments, one or more additional purification or processing steps are performed, including desalting, lyophilization, solvent exchange, or formulation into a desired composition.

The described systems and methods can be modified in any suitable manner. For instance, in some embodiments, one or more components of the conjugate 20 (e.g., the backbone 22, the shuttle 24, the tau-targeting ligand 26, or the tagging domain 28) can be substituted, modified, rearranged, omitted, or supplemented with additional components. For example, some embodiments use one or more oligothiophenes, anle138b, portions of the foregoing, or any other tau-targeting ligand. In some embodiments, one or more components are replaced with functionally equivalent components configured to perform similar roles, including alternative shuttles configured to engage different transport mechanisms, alternative ligands configured to bind different target molecules, or alternative tagging domains configured to facilitate degradation or clearance via different intracellular pathways. In some embodiments, one or more components are combined, split into multiple components, or arranged in different configurations (e.g., linear, branched, cyclic, or multi-domain architectures). In some embodiments, the conjugate includes multiple instances of one or more components (e.g., multiple tau-targeting ligands, multiple tagging domains, or multiple shuttles). In some embodiments, the conjugate includes additional domains configured to provide one or more functions, such as diagnostic, imaging, targeting, stabilizing, or delivery-related functions. In some embodiments, one or more components are configured to be selectively activatable, reversible, degradable, or otherwise dynamically responsive to one or more conditions. In some embodiments, the conjugate is incorporated into one or more pharmaceutical compositions, formulations, delivery systems, or dosage forms, including solutions, suspensions, tablets, capsules, sprays, or other suitable forms, and can be administered via any suitable route. Additionally, any of the components, features, embodiments, or methods described herein can be combined in any suitable manner, and any such combinations are expressly contemplated as part of the present disclosure.

In some embodiments, the system is configured to target a protein other than tau. For example, some embodiments are configured to selectively bind pathological variants of any of the following: amyloid-beta, alpha-synuclein, TDP-43 (TAR DNA-binding protein 43), FUS, huntingtin (mutant HTT), prion protein, neurofilament light chain, neurofilament heavy chain, ubiquitin, SOD1 (superoxide dismutase 1), C9orf72 dipeptide repeat proteins (e.g., poly-GA, poly-GR, poly-PR), beta-2 microglobulin, cystatin C, apolipoprotein E (ApoE), complement proteins (e.g., C1q, C3), GFAP (glial fibrillary acidic protein), myelin basic protein (MBP), synaptophysin, progranulin, presenilin-1, presenilin-2, LRRK2, DJ-1 (PARK7), ataxin proteins (e.g., ataxin-1, ataxin-2, ataxin-3), reelin, or any other pathological or potentially pathological proteins. Some embodiments are configured to target multiple proteins (e.g., by including multiple targeting domains).

In addition to the aforementioned features, the described systems and methods can include any other suitable feature. In some embodiments, the systems and methods provide advantages over prior approaches by integrating multiple functional domains into a single conjugate 20, thereby enabling coordinated transport, targeting, and clearance of pathological molecules within the central nervous system. For example, in some embodiments, the inclusion of the shuttle 24 enables active or facilitated transport of the conjugate across the blood-brain barrier (BBB), overcoming limitations associated with passive diffusion or poor brain penetration observed in certain prior art compounds. In some embodiments, the tau-targeting ligand 26 provides selective binding to pathological tau species, thereby reducing off-target effects associated with non-specific binding observed in certain small-molecule or antibody-based approaches.

In some embodiments, the tagging domain 28 enables inducible clearance of target molecules, such as tau proteins, by facilitating routing of the conjugate-target complex to intracellular degradation pathways (e.g., lysosomal or autophagic pathways). In some embodiments, this inducible functionality provides advantages over constitutively active degradation systems by reducing premature degradation or off-target effects, and by enabling degradation to occur preferentially in the presence of pathological targets. In some embodiments, the use of a masked or activatable tagging domain distinguishes the present systems and methods from prior art approaches that lack conditional activation or that rely on separate components to achieve targeting and degradation.

In some embodiments, the combination of transport, targeting, and inducible degradation within a single conjugate provides advantages over multi-component systems, including improved delivery efficiency, reduced complexity, and enhanced control over spatial and temporal aspects of therapeutic activity. In some embodiments, the described systems and methods enable targeting of intracellular aggregates that are not readily accessible to extracellular therapeutics (e.g., antibodies), thereby addressing limitations associated with therapies that do not effectively engage intracellular targets. In some embodiments, the systems and methods provide a platform that can be adapted to target different molecules, pathways, or disease states by modifying one or more components of the conjugate, thereby offering flexibility and broad applicability relative to prior art approaches.

Any and all of the components in the figures, embodiments (or example embodiments), implementations, instances, cases, methods, applications, iterations, and other parts of this disclosure can be combined in any suitable manner. Additionally, any component can be removed, separated from other components, modified with or without modification of like components, or otherwise altered together or separately from anything else disclosed herein.

As used herein, the singular forms “a”, “an”, “the” and other singular references include plural referents, and plural references include the singular, unless the context clearly dictates otherwise. For example, reference to a moiety includes reference to one or more moieties, and reference to residues includes reference to one or more residues. In addition, where reference is made to a list of elements (e.g., elements a, b, and c), such reference is intended to include any one of the listed elements by itself, any combination of less than all of the listed elements, and/or a combination of all of the listed elements. Moreover, the term “or” by itself is not exclusive (and therefore may be interpreted to mean “and/or”) unless the context clearly dictates otherwise. Similarly, the term “and” by itself is not exclusive (and therefore may be interpreted to mean “and/or”) unless the context clearly dictates otherwise. Furthermore, the terms “including”, “having”, “such as”, “for example”, “e.g.”, and any similar terms are not intended to limit the disclosure, and may be interpreted as being followed by the words “without limitation”.

In addition, as the terms “on”, “disposed on”, “attached to”, “connected to”, “coupled to”, etc. are used herein, one object (e.g., a material, element, structure, member, etc.) can be on, disposed on, attached to, connected to, or otherwise coupled to another object—regardless of whether the one object is directly on, attached, connected, or coupled to the other object, or whether there are one or more intervening objects between the one object and the other object. Also, directions (e.g., “front”, “back”, “on top of”, “below”, “above”, “top”, “bottom”, “side”, “up”, “down”, “under”, “over”, “upper”, “lower”, “lateral”, “right-side”, “left-side”, “base”, etc.), if provided, are relative and provided solely by way of example and for ease of illustration and discussion and not by way of limitation.

The described systems and methods may be embodied in other specific forms without departing from their spirit or essential characteristics. The described embodiments, examples, and illustrations are to be considered in all respects only as illustrative and not restrictive. The scope of the described systems and methods is, therefore, indicated by the appended claims rather than by the foregoing description. All changes that come within the meaning and range of equivalency of the claims are to be embraced within their scope. Moreover, any component and characteristic from any embodiments, examples, and illustrations set forth herein can be combined in any suitable manner with any other components or characteristics from one or more other embodiments, examples, and illustrations described herein.

### EXAMPLE EMBODIMENTS

Example 1: a therapeutic conjugate comprising: a peptide backbone; a shuttle configured to cause the therapeutic conjugate to cross a blood-brain barrier of a patient; a targeting ligand configured to bind to a protein; and a tagging domain configured to mark the therapeutic conjugate for degradation after the tau-targeting ligand has bound to the tau protein.

Example 2: the therapeutic conjugate of example 1, wherein the shuttle comprises a phosphocholine analog.

Example 3: the therapeutic conjugate of either of the foregoing examples, wherein the shuttle further comprises a lipid tail configured to engage a choline transporter.

Example 4: the therapeutic conjugate of any of the foregoing examples, wherein the lipid tail comprises a C16-C20 tail.

Example 5: the therapeutic conjugate of any of the foregoing examples, wherein the choline transporter is selected from the group consisting of CTL1 and CTL2.

Example 6: the therapeutic conjugate of any of the foregoing examples, wherein the phosphocholine analog comprises a quaternary-ammonium headgroup.

Example 7: the therapeutic conjugate of any of the foregoing examples, wherein the peptide backbone comprises an amino acid sequence consisting of between 10 and 50 amino acids.

Example 8: the therapeutic conjugate of any of the foregoing examples, wherein the peptide backbone comprises 4-fluoro-L-phenylalanine.

Example 9: the therapeutic conjugate of any of the foregoing examples, wherein the peptide backbone comprises at least 10 mol % 4-fluoro-L-phenylalanine.

Example 10: the therapeutic conjugate of any of the foregoing examples, wherein the targeting ligand is a tau-targeting ligand configured to bind pathological tau.

Example 11: the therapeutic conjugate of any of the foregoing examples, wherein the tau-targeting ligand comprises a hetero-tricyclic ligand.

Example 12: the therapeutic conjugate of any of the foregoing examples, wherein the tau-targeting ligand exhibits a dissociation constant of less than 100 nM for the tau protein.

Example 13: the therapeutic conjugate of any of the foregoing examples, wherein the tau protein comprises a hyperphosphorylated tau protein.

Example 14: the therapeutic conjugate of any of the foregoing examples, wherein the tagging domain comprises a masked targeting domain configured to be activated by a triggering event.

Example 15: the therapeutic conjugate of any of the foregoing examples, wherein the triggering event comprises cleavage of a spacer by a protease.

Example 16: the therapeutic conjugate of any of the foregoing examples, wherein the triggering event is configured to be induced by the therapeutic conjugate binding to the tau protein.

Example 17: the therapeutic conjugate of any of the foregoing examples, wherein activation of the tagging domain marks the therapeutic conjugate for lysosomal clearing.

Example 18: the therapeutic conjugate of any of the foregoing examples, wherein the tau-targeting ligand comprises 7-aza-benzothieno[3,2-c]quinolin-5-one.

Example 19: the therapeutic conjugate of any of the foregoing examples, wherein the tagging domain comprises a lysosomal motif comprising a Leu-Leu sequence.

Example 20: the therapeutic conjugate of any of the foregoing examples, wherein the Leu-Leu sequence is part of a Leu-Leu-Gly-Asp sequence.

Example 21: the therapeutic conjugate of any of the foregoing examples, wherein the tagging domain comprises a spacer.

Example 22: the therapeutic conjugate of any of the foregoing examples, wherein the spacer comprises a cathepsin-labile spacer.

Example 23: the therapeutic conjugate of any of the foregoing examples, wherein the amino acid sequence consists of between 18 and 22 amino acid residues.

Example 24: the therapeutic conjugate of any of the foregoing examples, comprising or formulated as a sterile aqueous solution.

Example 25: the therapeutic conjugate of any of the foregoing examples, wherein the therapeutic conjugate is present within an aqueous solution at 5-20 mg/mL.

Example 26: a pharmaceutical composition comprising the therapeutic conjugate of any of the foregoing examples.

Example 27: the pharmaceutical composition of any of the foregoing examples packaged in a metered spray.

Example 28: the pharmaceutical composition of any of the foregoing examples, the metered spray delivering approximately 0.1 mg per actuation.

Example 29: the pharmaceutical composition of any of the foregoing examples, comprising a microtablet.

Example 30: the pharmaceutical composition of any of the foregoing examples, the microtablet containing 1-5 mg of the conjugate.

Example 31: the pharmaceutical composition of any of the foregoing examples, the microtablet further comprising β-cyclodextrin.

Example 32: the pharmaceutical composition of any of the foregoing examples, the composition further comprising a pharmacologically acceptable intranasal carrier.

Example 33: a method of treating a disease using the pharmaceutical composition of any of the foregoing examples or the therapeutic conjugate of any of the foregoing examples.

Example 34: the method of any of the foregoing, wherein the disease is Alzheimer’s disease.

Example 35: the method of any of the foregoing, wherein the disease is a tauopathy.

Example 36: the method of any of the foregoing, comprising administration once daily for at least 14-30 days.

Example 37: the method of any of the foregoing, further comprising performing imaging tau aggregates.

Example 38: the method of any of the foregoing, wherein the imaging comprises any of PET imaging, MRI imaging, or CT imaging.

Example 39: the method of any of the foregoing, wherein the disease comprises at least one of frontotemporal dementia, chronic traumatic encephalopathy, traumatic brain injury, and long-COVID.

Example 40: the method of any of the foregoing, further comprising co-administration of at least one of dexamethasone and minocycline.

Example 41: the method of any of the foregoing, further comprising use of the conjugate for inhibiting aggregation of hyper-phosphorylated tau protein.

Example 42: the method of any of the foregoing examples further comprising monitoring cerebrospinal fluid biomarkers during treatment.

Example 43: use of the therapeutic conjugate of any of the foregoing examples for inhibiting aggregation of hyperphosphorylated tau protein.

Example 44: use of the therapeutic conjugate of any of the foregoing examples for promoting lysosomal clearance of tau aggregates.

Example 45: the therapeutic conjugate of any of the foregoing examples, wherein the phosphocholine analog is replaced by a glucose-mimic headgroup configured for GLUT1 transport.

Example 46: the therapeutic conjugate of any of the foregoing examples, wherein the tau-targeting ligand comprises an anle138b derivative selective for α-synuclein.

Example 47: a kit comprising the pharmaceutical composition of any of the foregoing examples and printed instructions for intranasal administration.

Example 48: a kit comprising the therapeutic conjugate of any of the foregoing examples, a PET imaging tracer selective for tau, and instructions for imaging-guided therapy.

Example 49: the therapeutic conjugate of any of the foregoing examples, wherein a calculated logP of the conjugate is between about 2.5 and about 4.0.

Example 50: the therapeutic conjugate of any of the foregoing examples further comprising a PEG4 linker between the peptide backbone and the lysosomal-targeting motif.

Example 51: the pharmaceutical composition of any of the foregoing examples further comprising a mucoadhesive polymer selected from the group consisting of hyaluronic acid and carbomer.

Example 52: a method of reducing neuro-inflammation comprising administering the therapeutic conjugate of any of the foregoing examples in combination with a non-steroidal anti-inflammatory agent.

Example 53: the therapeutic conjugate of any of the foregoing examples, wherein the peptide backbone comprises between about 15% and about 30% of its amino-acid residues as 4-fluoro-L-phenylalanine.

Example 54: the therapeutic conjugate of any of the foregoing examples, wherein the shuttle is configured to engage a carrier-mediated transport mechanism at the blood-brain barrier.

Example 55: the therapeutic conjugate of any of the foregoing examples, wherein the shuttle is configured to mimic a naturally occurring nutrient.

Example 56: the therapeutic conjugate of any of the foregoing examples, wherein the shuttle comprises a glucose-mimicking structure configured to engage a glucose transporter.

Example 57: the therapeutic conjugate of any of the foregoing examples, wherein the shuttle comprises a peptide configured to bind to a receptor selected from the group consisting of LRP1, transferrin receptor, and insulin receptor.

Example 58: the therapeutic conjugate of any of the foregoing examples, wherein the shuttle is configured to permit release of the therapeutic conjugate following transport across the blood-brain barrier.

Example 59: the therapeutic conjugate of any of the foregoing examples, wherein the peptide backbone comprises one or more non-natural amino acids selected to increase resistance to proteolytic degradation.

Example 60: the therapeutic conjugate of any of the foregoing examples, wherein the peptide backbone comprises a cyclized structure.

Example 61: the therapeutic conjugate of any of the foregoing examples, wherein the peptide backbone comprises a stapled peptide configuration.

Example 62: the therapeutic conjugate of any of the foregoing examples, wherein the peptide backbone comprises a branched architecture.

Example 63: the therapeutic conjugate of any of the foregoing examples, wherein the tau-targeting ligand is coupled to a side chain of a lysine residue.

Example 64: the therapeutic conjugate of any of the foregoing examples, wherein the tau-targeting ligand is coupled to the peptide backbone via a triazole linkage.

Example 65: the therapeutic conjugate of any of the foregoing examples, wherein the tau-targeting ligand is configured to bind beta-sheet-rich regions of tau aggregates.

Example 66: the therapeutic conjugate of any of the foregoing examples, wherein the tau-targeting ligand is configured to preferentially bind aggregated tau relative to monomeric tau.

Example 67: the therapeutic conjugate of any of the foregoing examples, wherein the tau-targeting ligand comprises a fluorescent or imaging-capable moiety.

Example 68: the therapeutic conjugate of any of the foregoing examples, wherein the tagging domain comprises an inducible lysosomal targeting motif.

Example 69: the therapeutic conjugate of any of the foregoing examples, wherein the tagging domain comprises a KFERQ-like motif.

Example 70: the therapeutic conjugate of any of the foregoing examples, wherein the tagging domain comprises a tyrosine-based sorting motif.

Example 71: the therapeutic conjugate of any of the foregoing examples, wherein the tagging domain comprises a protease-cleavable linker selected to be cleaved by a cathepsin.

Example 72: the therapeutic conjugate of any of the foregoing examples, wherein activation of the tagging domain is dependent on binding of the tau-targeting ligand to a tau protein.

Example 73: the therapeutic conjugate of any of the foregoing examples, wherein activation of the tagging domain is dependent on an intracellular pH condition.

Example 74: the therapeutic conjugate of any of the foregoing examples, wherein the tagging domain is configured to direct a conjugate–target complex to a lysosome without requiring ubiquitination of the target.

Example 75: the therapeutic conjugate of any of the foregoing examples, wherein the tagging domain is configured to promote autophagic uptake of the conjugate–target complex.

Example 76: the therapeutic conjugate of any of the foregoing examples, wherein the tagging domain is configured to recruit one or more adaptor proteins involved in intracellular trafficking.

Example 77: the therapeutic conjugate of any of the foregoing examples, wherein the conjugate comprises multiple tau-targeting ligands.

Example 78: the therapeutic conjugate of any of the foregoing examples, wherein the conjugate comprises multiple tagging domains.

Example 79: the therapeutic conjugate of any of the foregoing examples, wherein the conjugate comprises multiple shuttles.

Example 80: a pharmaceutical composition comprising the therapeutic conjugate of any of the foregoing examples, wherein the composition is configured for intranasal administration.

Example 81: a method of treating a tauopathy comprising administering the therapeutic conjugate of any of the foregoing examples to a subject in need thereof.

Example 82: the therapeutic conjugate of any of the foregoing examples, wherein the targeting domain is configured to bind one or more pathological variants of any of the following: amyloid-beta, alpha-synuclein, TDP-43 (TAR DNA-binding protein 43), FUS, huntingtin (mutant HTT), prion protein, neurofilament light chain, neurofilament heavy chain, ubiquitin, SOD1 (superoxide dismutase 1), C9orf72 dipeptide repeat proteins (e.g., poly-GA, poly-GR, poly-PR), beta-2 microglobulin, cystatin C, apolipoprotein E (ApoE), complement proteins (e.g., C1q, C3), GFAP (glial fibrillary acidic protein), myelin basic protein (MBP), synaptophysin, progranulin, presenilin-1, presenilin-2, LRRK2, DJ-1 (PARK7), ataxin proteins (e.g., ataxin-1, ataxin-2, ataxin-3), reelin, or any other pathological or potentially pathological proteins.

What is claimed is:

1. A therapeutic conjugate comprising:
a peptide backbone;
a shuttle configured to cause the uptake of the therapeutic conjugate via endogenous transport pathways to cross a blood-brain barrier of a patient;
a tau-targeting ligand configured to bind to a pathological tau protein; and
a tagging domain configured to mark the therapeutic conjugate for degradation after the tau-targeting ligand has bound to the pathological tau protein.

2. The therapeutic conjugate of claim 1, wherein the shuttle comprises a phosphocholine analog.

3. The therapeutic conjugate of claim 2, wherein the shuttle further comprises a lipid tail configured to engage a choline transporter.

4. The therapeutic conjugate of claim 3, wherein the lipid tail comprises a C₁₆-C₂₀ tail.

5. The therapeutic conjugate of claim 3, wherein the choline transporter is selected from the group consisting of CTL1 and CTL2.

6. The therapeutic conjugate of claim 2, wherein the phosphocholine analog comprises a quaternary-ammonium headgroup.

7. The therapeutic conjugate of claim 1, wherein the peptide backbone comprises an amino acid sequence consisting of between 10 and 50 amino acids.

8. The therapeutic conjugate of claim 1, wherein the peptide backbone comprises 4-fluoro-L-phenylalanine.

9. The therapeutic conjugate of claim 1, wherein the peptide backbone comprises at least 10 mol % 4-fluoro-L-phenylalanine.

10. The therapeutic conjugate of claim 1, wherein the tau-targeting ligand comprises a hetero-tricyclic ligand.

11. The therapeutic conjugate of claim 1, wherein the tau-targeting ligand exhibits a dissociation constant of less than 100 nM for the pathological tau protein.

12. The therapeutic conjugate of claim 11, wherein the pathological tau protein comprises a hyperphosphorylated tau protein.

13. The therapeutic conjugate of claim 1, wherein the tagging domain comprises a masked targeting domain configured to be activated by a triggering event.

14. The therapeutic conjugate of claim 13, wherein the triggering event comprises cleavage of a spacer by a protease.

15. The therapeutic conjugate of claim 13, wherein the triggering event is configured to be induced by the therapeutic conjugate binding to the pathological tau protein.

16. The therapeutic conjugate of claim 1, wherein activation of the tagging domain marks the therapeutic conjugate for lysosomal clearing.

17. A therapeutic conjugate comprising:
a peptide backbone;
a shuttle configured to facilitate localization at or near a biological barrier and to promote uptake into or across the barrier via endogenous transport pathways;
a target-binding ligand configured to bind a pathological intracellular protein; and
a tagging domain configured, upon activation, to direct a conjugate–protein complex to an intracellular degradation pathway, wherein the tagging domain is inactive prior to binding of the ligand to the target and is activated following binding and an intracellular condition.

18. The therapeutic conjugate of claim 17, wherein the pathological intracellular protein comprises a pathological tau protein.

19. The therapeutic conjugate of claim 17, wherein the pathological intracellular protein comprises a pathological α-synuclein protein.

20. The therapeutic conjugate of claim 17, wherein the pathological intracellular protein comprises a pathological TDP-43 protein.
